(19) United States
(12) Patent Application Publication
(10) Pub. No.: US 2017/0215904 A1
(43) Pub. Date: Aug. 3, 2017

(54) SPECIMEN RETRIEVAL DEVICE, METHOD AND KIT

(71) Applicant: PCT/CA2015/000542, Montreal (CA)

(72) Inventors: Seif El-Din WASSEF, Montreal (CA); Togas TULANDI, Montreal (CA)

(21) Appl. No.: 15/489,702

(22) Filed: Apr. 17, 2017

Related U.S. Application Data
(63) Continuation of application No. PCT/CA2015/000542, filed on Oct. 15, 2015.

(51) Int. Cl. A61B 17/22; A61B 17/00; A61B 17/32

(52) U.S. Cl. CPC A61B 17/22031; A61B 17/320016; A61B 17/00234; A61B 17/32002; A61B 2017/347

(57) ABSTRACT

A method and a device for secured retrieval of tissue from inside a patient body and a kit comprising the device. the device comprises a tube housing a movable cutter. The cutter controllably extends from the tube while allowing continued fluid communication. The device comprises a retractable enclosure with a supporting frame linked to an impermeable membrane comprising an opening. Amplitude of the supporting frame is controlled. The opening in the impermeable membrane is unseal when enclosure is expanded and, when the tissue is positioned within the enclosure, the opening is sealably closed in the impermeable membrane. The moveable cutter morcellates the tissue into tissue specimens when the opening in the impermeable membrane is sealably closed while amplitude of the supporting frame is reduced to compress the tissue specimens and the tissue specimens are able to transit in the fluid communication channel through a discharging aperture after morcellation.

### PRIORITY STATEMENT UNDER 35 U.S.C §0.119 (E) & 37 C.F.R. §0.1.78

This non-provisional patent application claims priority based upon the prior U.S. provisional patent application No. 62/065,047, filed Oct. 17, 2014, in the names of Seif El-Din Wassef and Togas Tulandi and is a continuation from PCT/CA2015/000542, filed Oct. 15, 2014 in the name of Tulandi & Wassef Medical Inc., both applications being herein incorporated by reference.

### TECHNICAL FIELD

The present invention is generally directed to a medical device, method and kit, more specifically, to a tissue removal and mechanical morcellation device, method and kit, for preventing leakage of severed tissues inside the body during morcellation.

### BACKGROUND

Minimally invasive surgical procedures have gained wide acceptance in the areas of general and gynecological surgery. Minimally invasive techniques have been developed for procedures on several organs including the heart, lung, and kidney. Generally, these procedures make use of one or more small incisions (or other openings) to access internal tissues, often through a cannula, trocar, or other access device. Gas insufflation or fluid distension may be used to enhance the available space within the internal surgical site, and the procedure is often directed with reference to an image provided by an endoscope, a microscope, or using a remote imaging modality such as fluoroscopy, ultrasound, or other endoscopic imaging. Compared to laparotomy, minimally invasive surgical procedures such as laparoscopy is associated with reduced patient trauma, less pain and rapid recovery.

During minimally invasive surgical procedures, such as morcellating within a body, severed tissues which may be of infected or cancerous may leak into the body. This leakage may pose complications, spread infected tissue or cause other problems.

Unfortunately, many surgical techniques are difficult to accomplish through laparoscopic cannulas or other minimally invasive access devices. The present invention as least partially addresses this problem.

### SUMMARY

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present invention is generally directed to a medical device, method and kit, more specifically, to a tissue removal and mechanical morcellation device, method and kit, for preventing leakage of severed tissues inside the body during morcellation.

In a first aspect, the invention provides for a morcellation device for preventing leakage of tissue in a patient's body during removal from the patient's body, comprising a tube having a first end for inserting into the patient's body; a sealable enclosure, at the first end of the tube, for receiving the tissue inside the patient's body when the sealable enclosure is unsealed; a cutter, at the first end of the tube, for morcellating the tissue into a severed tissue in the sealable enclosure when the sealable enclosure is sealed; and a channel coupled with the tube for transporting the severed tissue from the sealable enclosure outside of the patient's body.

In another aspect, the invention provides for a sealable and impermeable sheath for preventing leakage of severed tissue inside a patient's body during morcellation, detachably attached to a morcellation device.

In a second aspect, the invention provides for a method for preventing leakage of severed tissue inside a patient's body during morcellation, comprising incising at least one incision in the patient's body; inserting a grasper into the patient's body; grasping a tissue; expanding a sealable enclosure inside the patient's body; receiving the tissue inside the sealable enclosure; sealing the sealable enclosure; engaging a cutter to cut the tissue; and transporting a severed tissue outside the enclosure through a channel toward the outside of the patient's body.

In a third aspect, the invention provides for a morcellation kit, comprising: a morcellator, including a tube comprising a first end for inserting into a patient's body; a sealable enclosure, at the first end of the tube, for receiving a tissue inside the patient's body when the sealable enclosure is unsealed; a cutter, at the first end of the tube, for morcellating the tissue in the sealable enclosure when the sealable enclosure is sealed; a channel coupled with the tube for transporting a severed tissue from the sealable and collapsible enclosure outside of the patient's body; and a grasper.

In a fourth aspect, a device is provided for secured retrieval of tissue from inside a patient body. The device comprises a handle, a tube, a movable cutter, a retractable enclosure, an impermeable membrane comprising an opening and a frame actuator mechanism. The handle is for controlling the device and is configured for manipulation from outside of the patient body. The handle comprises a specimen discharging aperture towards the outside of the patient body. The tube defines a fluid communication channel between an inward end and an outward end extending towards the specimen discharging aperture of the handle. The tube comprises an external surface configured on at least a portion thereof to interface between the inside and the outside of the patient body therealong. The movable cutter, housed within the tube, is controllably extendable from the inward end of the tube while allowing continued fluid communication in the fluid communication channel. The retractable enclosure is at least partly housed within the tube and comprises a supporting frame that defines a first retracted configuration and a second expanded configurations, wherein the first retracted configuration defines a first enclosure volume and hinders the fluid communication in the fluid communication channel. The impermeable membrane is linked to the supporting frame. The frame actuator mechanism controls amplitude of the supporting frame between the first retracted configuration and the second expanded configuration and vice-versa. The retractable enclosure, in the second expanded configuration, extends from the inward end of the tube and has a variable second enclosure volume, greater than or equal to the first enclosure volume, defined by the frame actuator mechanism upon controlling the amplitude of the supporting frame. The sealing mechanism, for controllably sealing the opening in the impermeable membrane, is configured to unseal the opening when the second enclosure volume is greater than the first enclosure volume and, when the tissue is positioned within the enclosure, sealably close the opening in the impermeable membrane, thereby sealing the retractable enclosure. The retractable enclosure receives the moveable cutter for morcellating the tissue into tissue specimens when the opening in the impermeable membrane is sealably closed, whereby the tissue specimens are able to transit in the fluid communication channel towards the discharging aperture after morcellation. The frame actuator mechanism reduces the amplitude of the supporting frame causing the tissue specimens to be compressed by the supporting frame during transition from the second expanded configuration to the first retracted configuration, the tissue specimens being discharged through the fluid communication channel towards the discharging aperture.

The sealing mechanism may optionally comprise a zipper-like mechanism.

The device may further comprise a tissue collection reservoir for receiving the severed tissue through the specimen discharging aperture on the outside of the patient's body.

The tissue specimens may be compressed by the supporting frame and the impermeable membrane. The tissue specimens may also be compressed by the supporting frame while the impermeable membrane defines a membrane volume greater than the second volume.

Optionally, the sealing mechanism may unseal the opening only when the second enclosure volume is greater than the first enclosure volume.

In some optional embodiments, the sealing mechanism, when the second enclosure volume is equal to the first enclosure volume, maintains closed the opening in the impermeable membrane and prevents unsealing of the opening in the impermeable membrane.

In a firth aspect, a method is provided for secured retrieval of tissue from inside a patient body. The method comprises inserting a device from outside of the patient body, the device comprising a specimen discharging aperture towards the outside of the patient body and a tube defining a fluid communication channel between an inward end and an outward end extending towards the specimen discharging aperture, the tube comprising an external surface configured on at least a portion thereof to interface between the inside and the outside of the patient body therealong. The method also comprises, from the device, expanding a retractable enclosure inside the patient's body, the retractable enclosure being at least partly housed within the tube and comprising an impermeable membrane comprising an opening. Expanding of the retractable enclosure comprises extending a supporting frame from the inward end of the tube into an expanded configuration from a retracted configuration, wherein the retracted configuration defines a first enclosure volume and hinders the fluid communication in the fluid communication channel and wherein the expanded configuration has a variable second enclosure volume, greater than or equal to the first enclosure volume, defined by the frame actuator mechanism upon controlling the amplitude of the supporting frame. The method also comprises unsealing the opening when the second enclosure volume is greater than the first enclosure volume and, when the tissue is positioned within the enclosure, sealably closing the opening in the impermeable membrane, thereby sealing the retractable enclosure the method than also comprises controllably extending a movable cutter, housed within the tube, from the inward end of the tube while allowing continued fluid communication in the fluid communication channel, wherein the moveable cutter morcellates the tissue into tissue specimens when the opening in the impermeable membrane is sealably closed, whereby the tissue specimens are able to transit in the fluid communication channel towards the discharging aperture after morcellation. The method yet further comprises reducing the amplitude of the supporting frame causing the tissue specimens to be compressed by the supporting frame during transition from the second expanded configuration to the first retracted configuration, the tissue specimens being discharged through the fluid communication channel towards the discharging aperture.

Optionally, sealing and unsealing may be performed using a zipper-like sealing mechanism.

The method may optionally further comprise discharging the severed tissue into a tissue collection reservoir through the specimen discharging aperture on the outside of the patient's body.

In some embodiments, the tissue specimens are compressed by the supporting frame and the impermeable membrane. The tissue specimens may also be compressed by the supporting frame while the impermeable membrane defines a membrane volume greater than the second volume.

Optionally, unsealing the opening may be performed only when the second enclosure volume is greater than the first enclosure volume.

In some embodiments, when the second enclosure volume is equal to the first enclosure volume, the opening in the impermeable membrane is maintained closed and prevented from unsealing.

The device described in relation to the fourth aspect may be used in accordance with the method described in the fifth aspect.

In a sixth aspect, a kit is provided for secured retrieval of tissue from inside a patient body. The kit comprises the device as defined herein above, including its different options, with regards to the fourth aspect. The kit also comprises a grasper. The grasper may be used to position the tissue in the enclosure. In some embodiments, the device and the grasper are disposable. In some embodiments, only the grasper is disposable and the device is reusable. In some embodiments, only the grasper is reusable and the device is disposable. In some embodiments, the grasper and the device are reusable.

The kit may be used in accordance with the method described in the fifth aspect.

### BRIEF DESCRIPTION OF THE DRAWINGS

Further features and exemplary advantages of the present invention will become apparent from the following detailed description, taken in conjunction with the appended drawings, in which:

FIG. 1 is a perspective view of an exemplary morcellator with an exemplary closed bag in accordance with the teachings of the present invention;

FIG. 2 shows a closer perspective view of the closed bag of the morcellator in FIG. 1, in accordance with the teachings of the present invention;

FIG. 3 shows a perspective view of an exemplary cutting tube of the morcellator which travels to its maximum stroke, in accordance with the teachings of the present invention;

FIG. 4 shows a perspective view of an exemplary sheath closure slider opened to its maximum (inner end) in the morcellator, in accordance with the teachings of the present invention;

FIG. 5 shows a perspective view of an exemplary opened bag after expansion due to the retracted cutter tube in the morcellator, in accordance with the teachings of the present invention;

FIG. 6 shows a perspective view of a mass located inside an exemplary bag cavity area of the morcellator, in accordance with the teachings of the present invention;

FIG. 7 shows a perspective view of the closed bag of the morcellator with a mass inside with a perspective view of an exemplary closure sliding mechanism, in accordance with the teachings of the present invention;

FIG. 8 shows a perspective view of the morcellator with a mass in the closed bag, in accordance with the teachings of the present invention;

FIG. 9 shows a perspective view of an exemplary retracted cage of the morcellator confining the mass, in accordance with the teachings of the present invention;

FIG. 10 shows a perspective view of the morcellator with the confined mass, in accordance with the teachings of the present invention;

FIG. 11 shows a perspective view of an exemplary maximum cutter stroke of the exemplary morcellator through the confined mass, in accordance with the teachings of the present invention;

FIG. 12 shows the reduced mass size in the morcellator after two exemplary full cutter strokes, in accordance with the teachings of the present invention;

FIG. 13 shows a perspective view of the morcellator after mass reduction, and a view of morsels extracted to an exemplary reservoir, in accordance with the teachings of the present invention;

FIG. 14 shows a perspective view of the final reduced size of the mass after a few cutter strokes of the morcellator, in accordance with the teachings of the present invention;

FIG. 15 shows a perspective view of the morcellator reaching an exemplary final operation stage and ready to be pulled out of the access port, in accordance with the teachings of the present invention;

FIG. 16 shows a perspective view of an exemplary bag structure showing an exemplary inner crest of the cutting tube, in accordance with the teachings of the present invention;

FIG. 17 shows a perspective view of an exemplary zipper-like closing mechanism, bag and cage of the morcellator, in accordance with the teachings of the present invention;

FIG. 18 shows a perspective view of the cutting tube as located inside the cage extension tube in the morcellator, and an exemplary housing tube, in accordance with the teachings of the present invention;

FIG. 19 shows a perspective view of the closing mechanism interacting with the housing tube, with the bag being pulled into an exemplary gap between the cage extension tube and the housing tube, in accordance with the teachings of the present invention;

FIG. 20 shows a perspective view of an exemplary morcellator configuration when wrapped into an exemplary introducing sleeve, in accordance with the teachings of the present invention;

FIG. 21 shows a perspective view of an exemplary morcellator's end when the bag is pulled inside the housing tube with the opening fully closed and sealed, in accordance with the teachings of the present invention;

FIG. 22 shows a perspective view of the exemplary morcellator's end showing a simple opening in the bag, in accordance with the teachings of the present invention;

FIG. 23 shows a perspective view of an exemplary o-ring located on the cage extension tube and in front of an exemplary bag ring of the morcellator, in accordance with the teachings of the present invention;

FIG. 24 shows a perspective view of the bag of the morcellator when inflated, in accordance with the teachings of the present invention;

FIG. 25 shows a perspective view of the cage of the morcellator with two exemplary different diameters, with an exemplary opening located on the smaller diameter portion of the cage, in accordance with the teachings of the present invention; and

FIG. 26 shows a flow chart for an exemplary method of preventing leakage of severed tissue inside a patient's body during morcellation, in accordance with the teachings of the present invention.

### DETAILED DESCRIPTION

It is sometimes desirable to remove relatively large masses of tissue, for example, to remove a kidney, a partial lung resection, a large myomatous uterus or myomas.

In order to allow removal of large tissue by laparoscopy, specialized devices have been used to sever large tissue masses into small segments, which are more easily removed. These devices generally include a rotating tube having a sharpened distal end which extends through a fixed outer tube. This sharpened end is inserted into a cavity such as an abdominal cavity of the patient through a cannula, or directly through an incision. The surgeon inserts a grasping device (such as endoscopic forceps or a laparoscopic grasper) through the rotating tube. Grasping a large mass of tissue to be removed, the surgeon retracts the tissue up into the tube, so that the rotating edge severs the grasped portion from the large mass. The size of the severed tissue is generally limited by the outline of the rotating edge, so that the surgeon can continue to retract the severed tissue out of the patient through the rotating tube. By repeating the grasping and severing procedure, surgeons can remove relatively large masses of tissue quite quickly. As the large tissue mass is removed in small, individually grasped morsels, these devices are often referred to as “morcellators”.

Although rotating tube morcellators represent a significant advancement in minimally invasive surgical procedures for removing large tissue masses, these known devices still have several significant drawbacks. First, some of non-disposable devices are relatively large, heavy, and expensive. Sterilizing these devices is fairly time consuming, and sliding motion between the tubes and dulling of the cutting edge limits their useful life. In addition, when removing the tissue masses, many small morsels can be left behind. This is particularly concerning in case of malignant tumors.

In light of the above, it is necessary to develop improved devices for removing tissues from internal surgical sites. It would be particularly desirable if these improved devices were adaptable for use with known laparoscopic and other minimally invasive surgical techniques. Importantly, those improved devices should be able to remove tissues while minimizing traces of it left behind, thereby enhancing the surgeon's control over the tissue removal procedure.

Exemplary embodiments will be described to demonstrate the use, principles, and function of the invention disclosed herein. These descriptions and illustrations are non-limiting exemplary embodiments and no limitation to the scope of the invention is thereby intended. Any alteration or modification to the device or alternative application of the invention principles are contemplated to normally occur by those with ordinary skill in the art to which the invention relates.

In one aspect, the invention provides for a morcellation device for preventing leakage of tissue in a patient's body during removal from the patient's body, comprising a tube comprising a first end for inserting into the patient's body; a sealable enclosure, at the first end of the tube, for receiving the tissue inside the patient's body when the sealable enclosure is unsealed; a cutter, at the first end of the tube, for morcellating the tissue into a severed tissue in the sealable enclosure when the sealable enclosure is sealed; and a channel coupled with the tube for transporting the severed tissue from the sealable enclosure outside of the patient's body.

Optionally, in the morcellation device, the cutter is a movable cutter for stroking through at least one of the tissue or the severed tissue inside the sealable enclosure. The cutter may also be stationary within the device. The cutter may be movable in a variety of way including rotationally, longitudinally and axially. The cutter may be automatically or manually operable.

Optionally, in the morcellation device, the sealable enclosure comprises a collapsible structure for maintaining a volume inside the patient's body for morcellating therein. The collapsible enclosure may be movable in a variety of ways including rotationally, longitudinally and axially, and may be automatically or manually operable.

Optionally, in the morcellation device, the tube is rotationally fixed to a body of the morcellation device. The tube may be capable of moving in a variety of ways including axially.

Optionally, in the morcellation device, the sealable enclosure comprises an opening and closing mechanism, for sealing and unsealing the sealable enclosure. The opening and closing mechanism may consist of a zipper-like fastener. Optionally, when the opening and closing mechanism partially pulls the sealable enclosure inside the tube, an opening in the sealable enclosure is sealed. The sealable enclosure may take on a variety of shapes and may be sealable in a variety of ways.

Optionally, in the morcellation device, a control mechanism is present for varying the volume and length of the sealable enclosure. The control mechanism may be manual or automatically operable.

Optionally, in the morcellation device, the sealable enclosure is inflatable for varying the volume of the sealable enclosure, keeping the sealable enclosure away from at least one of the cutter and the movable cutter. The inflation may be enabled by the use of pressurized CO₂.

Optionally, when the morcellation device includes a movable cutter, the sealable enclosure may comprise a collapsible structure for maintaining a volume inside the patient's body for morcellating therein, and wherein when the movable cutter extends toward a maximum stroke, the sealable enclosure is collapsed along a longitudinal axis of the tube, for inserting into and removing from the patient's body.

Optionally, when the movable cutter retracts toward a minimum stroke, the sealable enclosure increases in volume.

Optionally, the morcellation device has a sealable enclosure that expands to a sphere-like shape.

Optionally, when the sealable enclosure decreases in volume, a compression force occurs for impeding movement of at least one of the tissue and the severed tissue.

Optionally, when the cutter morcellates the tissue, the severed tissue is transported out of the sealable enclosure through the channel, toward the outside of the patient's body. The severed tissue may be transported out of the sealable enclosure by a force initiated by the movable cutter.

Optionally, if the sealable enclosure is collapsible, when the severed tissue reaches a size compatible with a diameter of the channel, the sealable enclosure retracts inside the tube. Furthermore, the sealable enclosure may comprise a locking mechanism for allowing the sealable enclosure to hold volume during morcellation. The locking mechanism may function with the use of a spring system. The locking mechanism may operate to lock the sealable enclosure once it is extended fully, since there will be a retracting force exerted by the spring(s), which would reach max force at this point. In order for the enclosure not to ‘sling shot’ backwards into the outer tube, the lock may be included in the exemplary morcellator.

Optionally, when the severed tissue reaches a size compatible with a diameter of the channel, the tube extends to cover the sealable enclosure. The extension may occur in a variety of ways including automatically and manually.

Optionally, the morcellation device may further comprise at least one actuator, on the body of the morcellation device, for operating the movable cutter or the sealable enclosure. The enclosure may be collapsible. The actuator may trigger an automatic mode, or a manual mode, capable of overriding the automatic mode. For instance, it may be desirable to proceed with automatic morcellation for a complete procedure or for a limited period during the procedure. The actuator, or one or more buttons or trigger, may be used to toggle or otherwise select the automatic or manual morcellation modes. The actuator may be of variable location on the device, including on a handle.

Optionally, the sealable enclosure may comprise a cage-like structure for expanding and retracting inside a patient's body, and a sealable and impermeable sheath for preventing leakage of the severed tissue inside the patient's body during morcellation. The cage-like structure and the sealable and impermeable sheath may be covered by a removable sleeve for securing the sealable enclosure before insertion into the patient's body. The removable sleeve may be removed from the morcellation device before introduction into the patient's body. Just like many of the features described herein, the removable sleeve may be coupled with other embodiments of the sealable enclosure.

Optionally, the morcellation device may comprise at least one tissue collection reservoir for receiving the severed tissue on the outside of the patient's body. The tissue collection reservoir may comprise an aperture for allowing access therein from the outside. The aperture may comprise a removable lid. The tissue collection reservoir may be removable and disposable.

Optionally, at least a portion of the morcellation device is disposable. The entire device may be disposable. Parts of the morcellation device may be exchanged with new parts of the morcellation device during a procedure.

In another aspect, the invention provides for a sealable and impermeable sheath for preventing leakage of severed tissue inside a patient's body during morcellation, detachably attached to a morcellation device. The sealable and impermeable sheath may take on a variety of forms and may be operable with or without an enclosure-type structure. The sealable and impermeable sheath may be disposable. The sealable and impermeable sheath may be sealed and opened in a variety of ways.

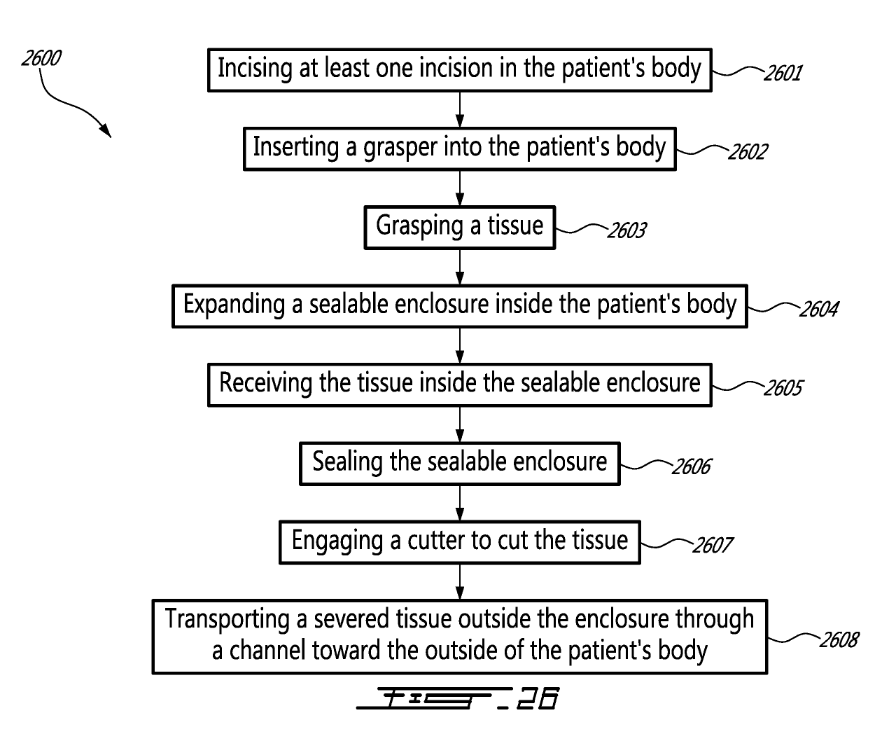

In another aspect, the invention provides a method for preventing leakage of severed tissue inside a patient's body during morcellation, comprising incising at least one incision in the patient's body; inserting a grasper into the patient's body; grasping a tissue; expanding a sealable enclosure inside the patient's body; receiving the tissue inside the sealable enclosure; sealing the sealable enclosure; engaging a cutter to cut the tissue; and transporting a severed tissue outside the enclosure through a channel toward the outside of the patient's body, as will be better defined with particular reference to FIG. 26.

Optionally, the method comprises transporting the tissue from the sealable enclosure into a tissue collection reservoir. The transport of tissue may be enabled by a variety of forces.

Optionally, the method comprises a cutter which is a movable cutter for stroking through at least one of the tissue or the severed tissue inside the sealable enclosure. The cutter may be automatically or manually operable.

Optionally, the method operates such that the cutter is a stationary cutter for morcellating at least one of the tissue or the severed tissue held inside the sealable enclosure by the grasper.

Optionally, the method operates such that pressurized CO₂ is used to expand the sealable enclosure.

Optionally, the method comprises a tube which is rotationally fixed to a body of the morcellation device.

Optionally, the method comprises a sealable enclosure having an opening and closing mechanism, for sealing and unsealing the sealable enclosure.

Optionally, the method operates such that wherein the opening and closing mechanism consists of a zipper-like fastener. When a tube is rotationally fixed to a body of the morcellation device, the opening and closing mechanism may partially pull the sealable enclosure inside the tube, sealing an opening in the sealable enclosure.

Optionally the method comprises a control mechanism for varying the volume and length of the sealable enclosure. The control mechanism may be manually or automatically operable.

Optionally, the method operates such that the sealable enclosure is inflatable for varying the volume of the sealable enclosure.

Optionally, when the method comprises a movable cutter, the sealable enclosure may comprise a collapsible structure for maintaining a volume inside the patient's body for morcellating therein, and the movable cutter may extend toward a maximum stroke as the sealable enclosure is collapsed along a longitudinal axis of the tube, for inserting into and removing from the patient's body. Optionally, the movable cutter retracts toward a minimum stroke, while the sealable enclosure increases in volume.

Optionally, the method operates such that the sealable enclosure expands to a sphere-like shape.

Optionally, the method operates such that when the sealable enclosure decreases in volume, a compression force occurs for impeding movement of at least one of the tissue and the severed tissue.

Optionally, the method operates such that the severed tissue is transported out of the sealable enclosure through the channel, toward the outside of the patient's body. The severed tissue may be transported out of the sealable enclosure by a force initiated by the movable cutter.

Optionally, when the severed tissue reaches a size compatible with a diameter of the channel, the sealable enclosure retracts inside the tube. This function may operate to compress the tissue inside the sealable enclosure.

Optionally, the method operates such that when the severed tissue reaches a size compatible with a diameter of the channel, the tube extends to cover the sealable enclosure. This function may operate to compress the tissue inside the sealable enclosure.

Optionally, the method operates such that the sealable enclosure comprises a locking mechanism for allowing the sealable enclosure to hold volume during morcellation. The locking mechanism may function with the use of a spring system.

Optionally, the method operates such that a tube is rotationally fixed to a body of the morcellation device, further comprising at least one actuator, on the body of the morcellation device, for operating the movable cutter or the sealable enclosure.

Optionally, the method may operate with one or several triggers or actuators, which may be automatically or manually operable. The actuators may operate a number of mechanisms, for example the cutter or the sealable enclosure. Optionally, the at least one actuator triggers an automatic mode. Optionally, the at least one actuator triggers a manual mode, capable of overriding the automatic mode. The actuator may be of variable location on the device, including on a handle.

Optionally, in the method, the sealable enclosure may comprise a cage-like structure for expanding and retracting inside a patient's body, and a sealable and impermeable sheath for preventing leakage of the severed tissue inside the patient's body during morcellation. The cage-like structure and the sealable and impermeable sheath may be covered by a removable sleeve for securing the sealable enclosure before insertion into the patient's body. The removable sleeve may be removed from the morcellation device before introduction into the patient's body. A removable sleeve may be coupled with other embodiments of the sealable enclosure.

Optionally, the invention may comprise at least one tissue collection reservoirs for receiving the severed tissue on the outside of the patient's body. The reservoir may be removable and disposable. The tissue collection reservoir may comprise an aperture for allowing access therein. The tissue collection reservoir may be removable and/or disposable. The invention may comprise more than one tissue collection reservoir.

In another aspect, the invention provides a morcellation kit, comprising: a morcellator, including a tube comprising a first end for inserting into a patient's body; a sealable enclosure, at the first end of the tube, for receiving a tissue inside the patient's body when the sealable enclosure is unsealed; a cutter, at the first end of the tube, for morcellating the tissue in the sealable enclosure when the sealable enclosure is sealed; a channel coupled with the tube for transporting a severed tissue from the sealable and collapsible enclosure outside of the patient's body; and a grasper.

Optionally, the sealable feature of the morcellator may operate with a balloon, which can be inflated to impede leakage of tissues from an enclosure within the body.

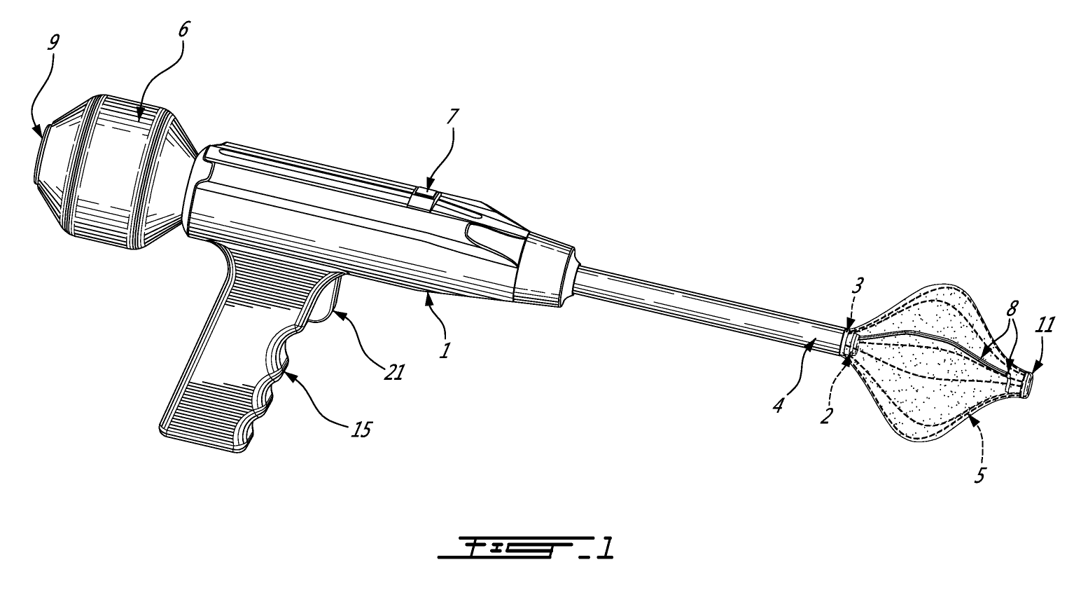

Reference is now made concurrently to FIGS. 1 to 26, which show different embodiments of the exemplary morcellation device in accordance with the present invention. Skilled person will understand that the depicted example is provided for ease of understanding the concepts of the present invention and not to limit the scope of the appended claims.

FIG. 1 is a perspective view of the exemplary morcellator with a closed bag in accordance with the teachings of the present invention.

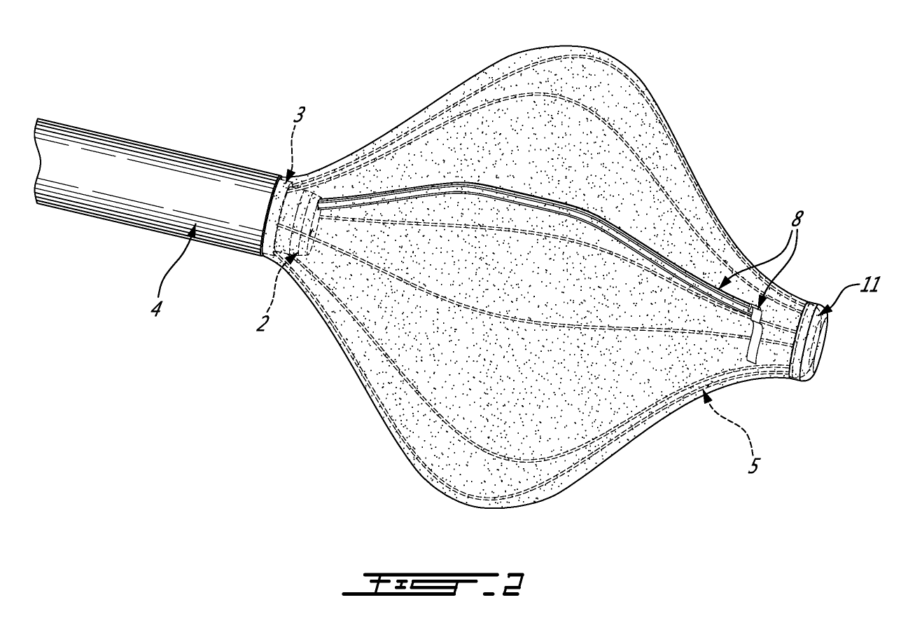

FIG. 2 shows a closer perspective view of the closed bag of the exemplary morcellator in FIG. 1, in accordance with the teachings of the present invention.

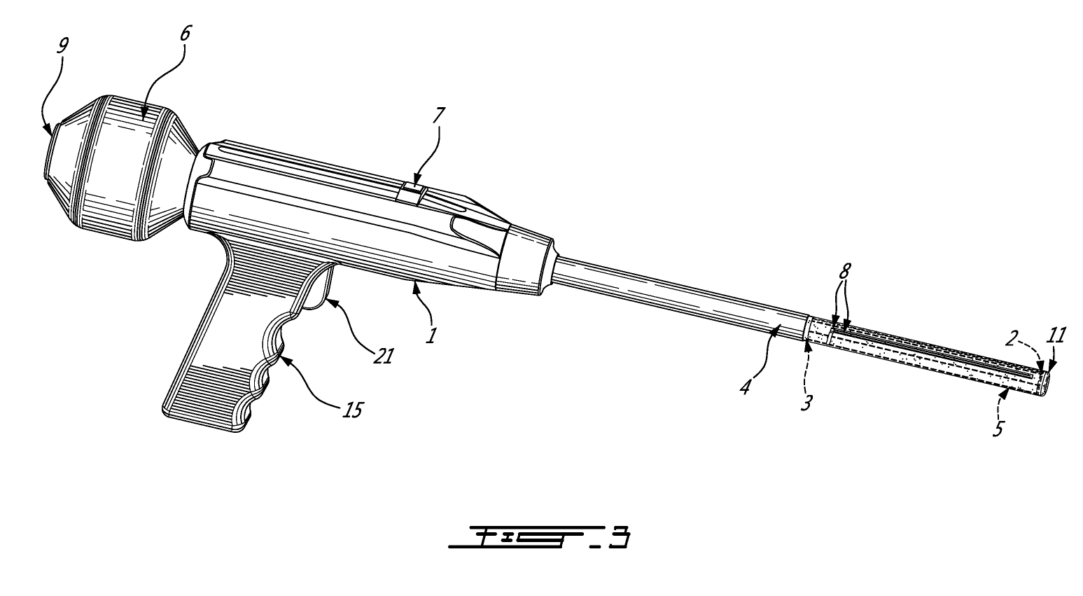

FIG. 3 shows a perspective view of the cutting tube of the exemplary morcellator which travels to its maximum stroke (130 mm) stretching the cage flat while the cage is at its maximum position, in accordance with the teachings of the present invention.

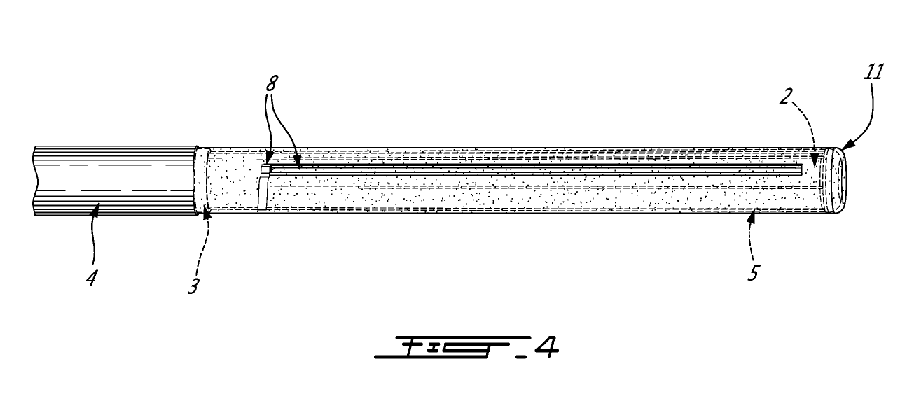

FIG. 4 shows a perspective view of the exemplary morcellator's sheath closure slider opened to its maximum (inner end), in accordance with the teachings of the present invention.

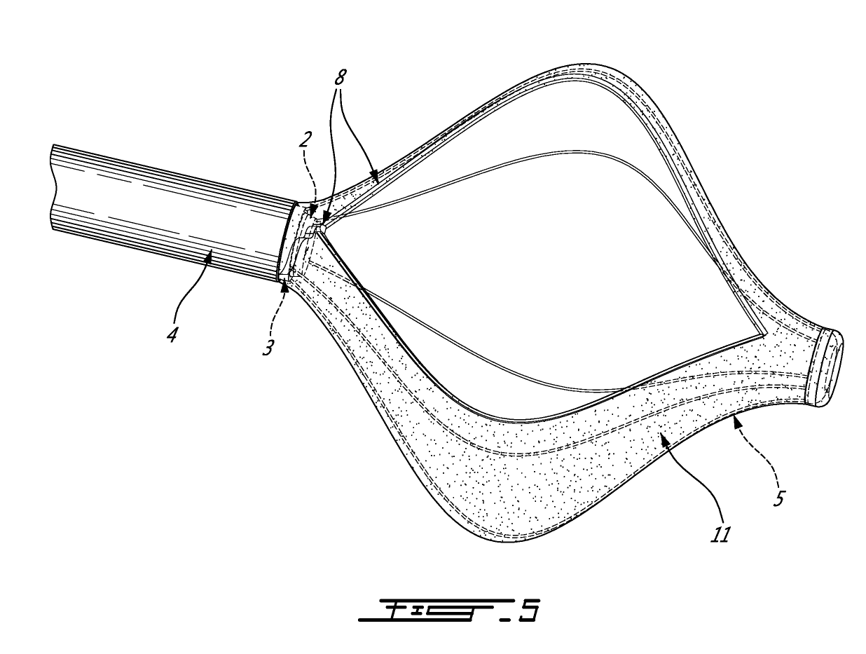

FIG. 5 shows a perspective view of the exemplary morcellator's opened bag after expansion due to the retracted cutter tube, in accordance with the teachings of the present invention.

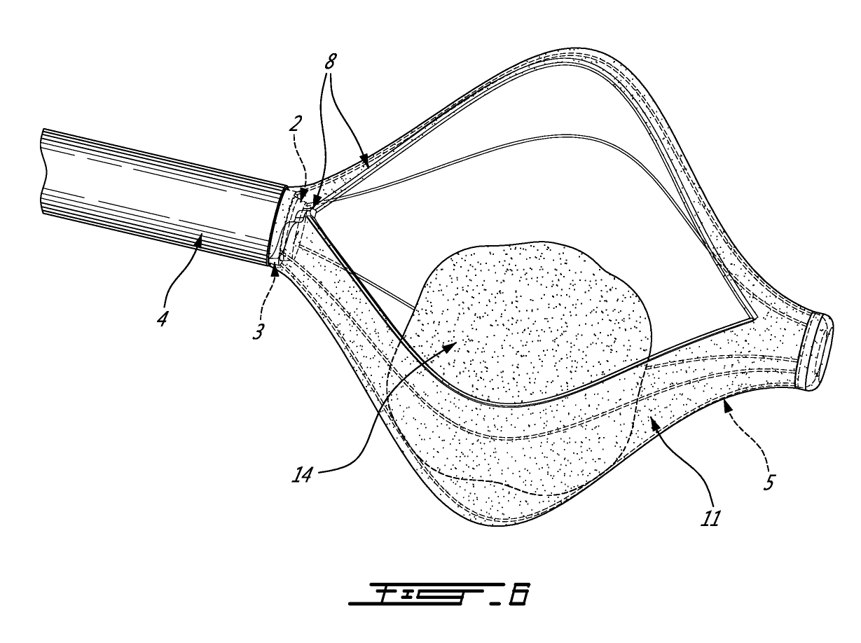

FIG. 6 shows a perspective view of a mass located inside the exemplary bag's cavity area in accordance with the teachings of the present invention.

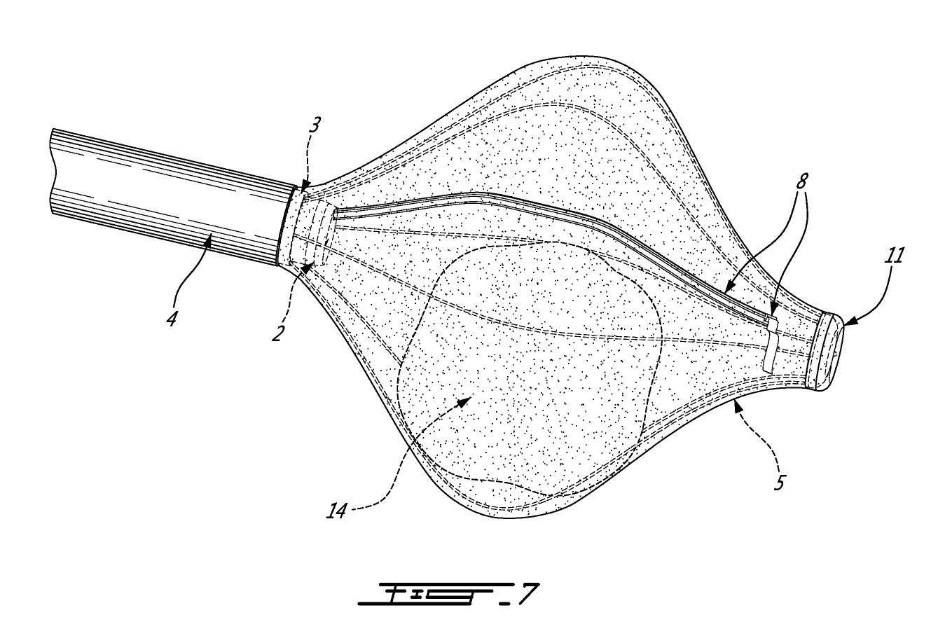

FIG. 7 shows a perspective view of the closed bag of the exemplary morcellator with a mass inside with a perspective view of a closure sliding mechanism, in accordance with the teachings of the present invention.

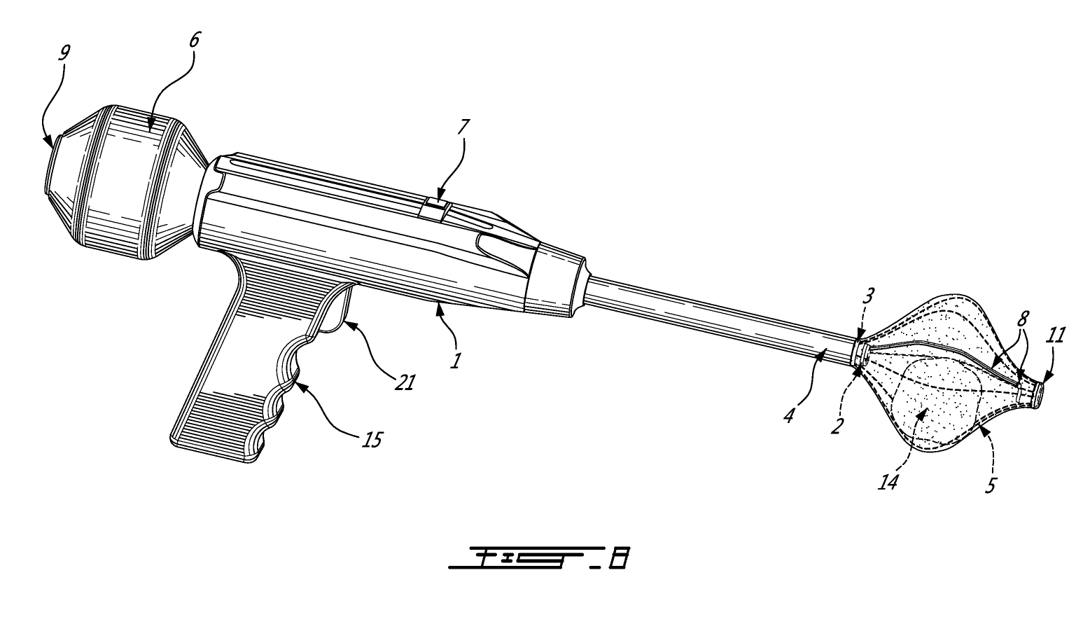

FIG. 8 shows a perspective view of the exemplary morcellator with a mass in closed bag, in accordance with the teachings of the present invention.

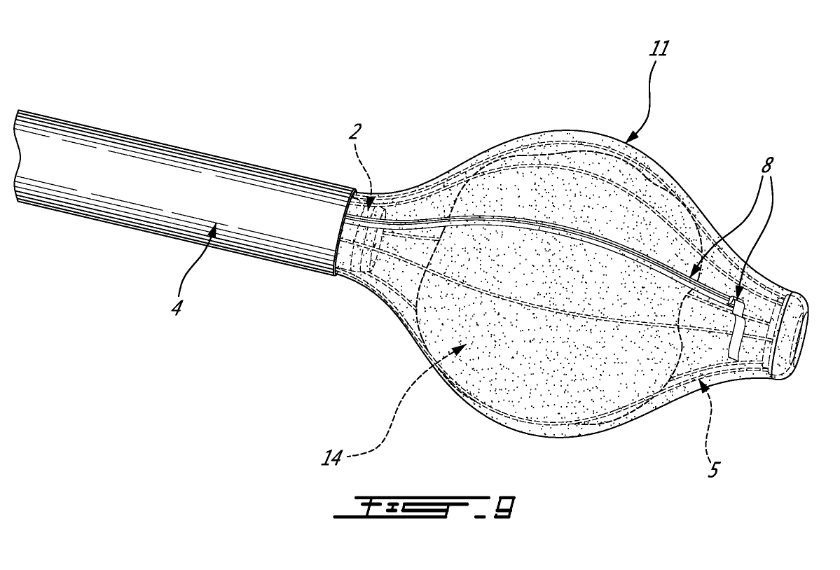

FIG. 9 shows a perspective view of a retracted cage of the exemplary morcellator confining the mass, in accordance with the teachings of the present invention.

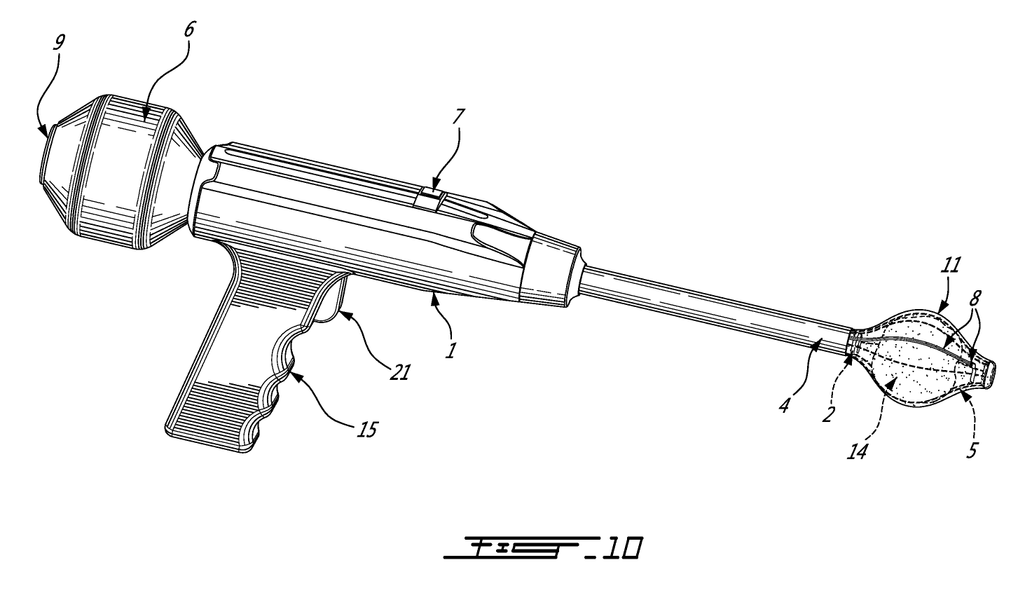

FIG. 10 shows a perspective view of the exemplary morcellator with the confined mass, in accordance with the teachings of the present invention.

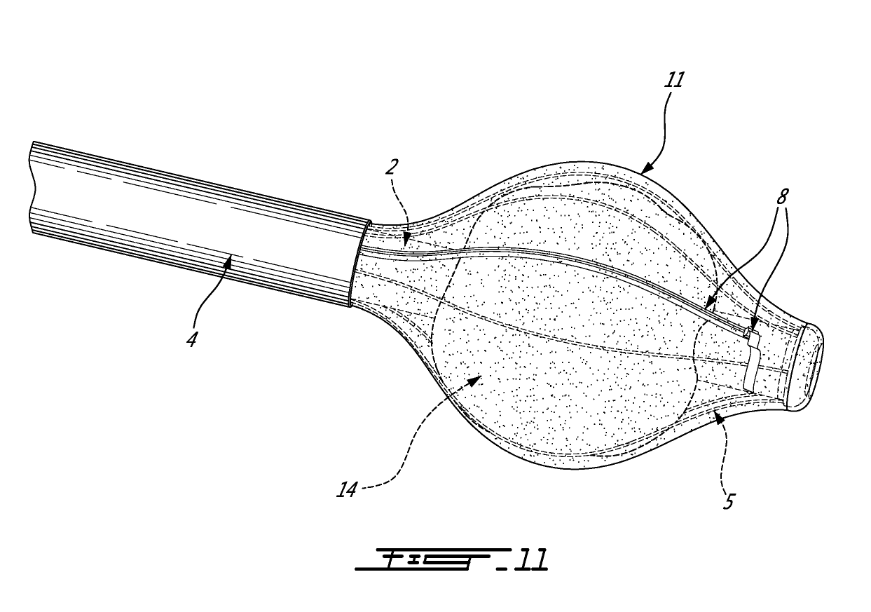

FIG. 11 shows a perspective view of the maximum cutter stroke of the cutter in the exemplary morcellator through the confined mass, in accordance with the teachings of the present invention.

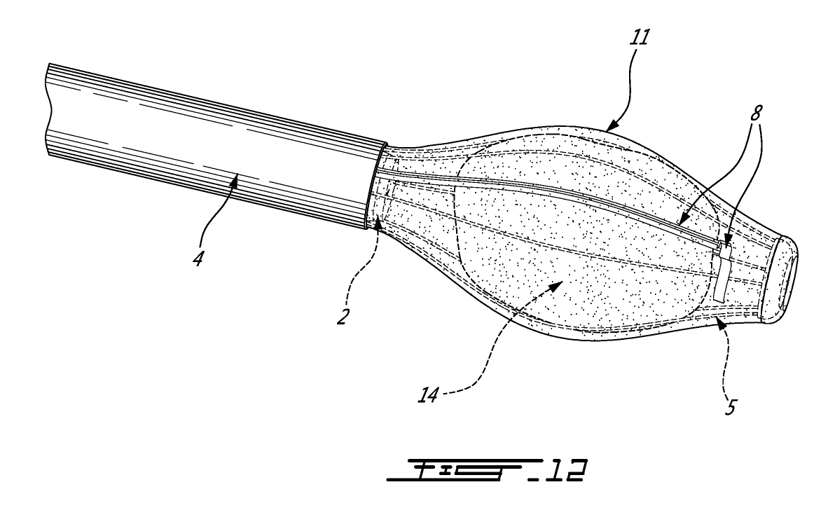

FIG. 12 shows the reduced mass size in the exemplary morcellator after two full cutter strokes, in accordance with the teachings of the present invention.

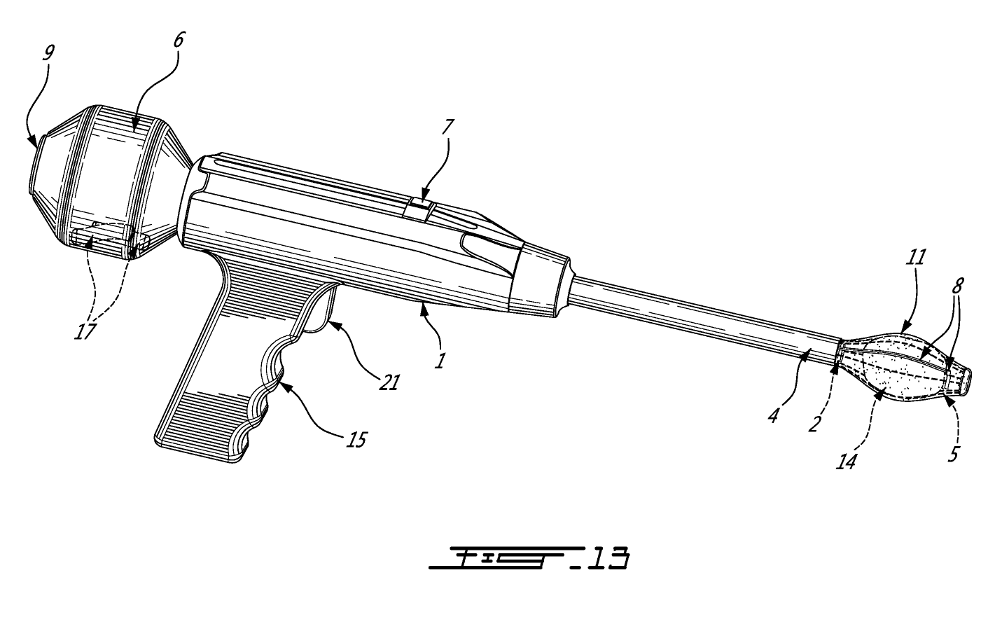

FIG. 13 shows a perspective view of the exemplary morcellator after mass reduction, and a view of morsels extracted to the reservoir, in accordance with the teachings of the present invention.

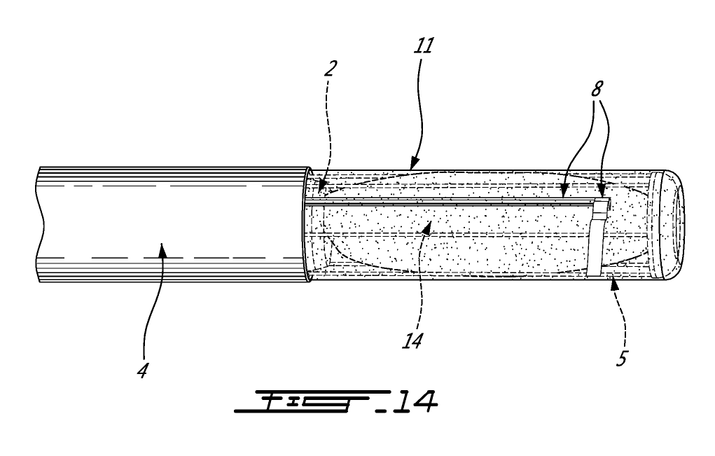

FIG. 14 shows a perspective view of the final reduced size of the mass after a few cutter strokes, wherein the mass is small enough to safely pull the exemplary morcellator out of the access port ending the procedure, in accordance with the teachings of the present invention.

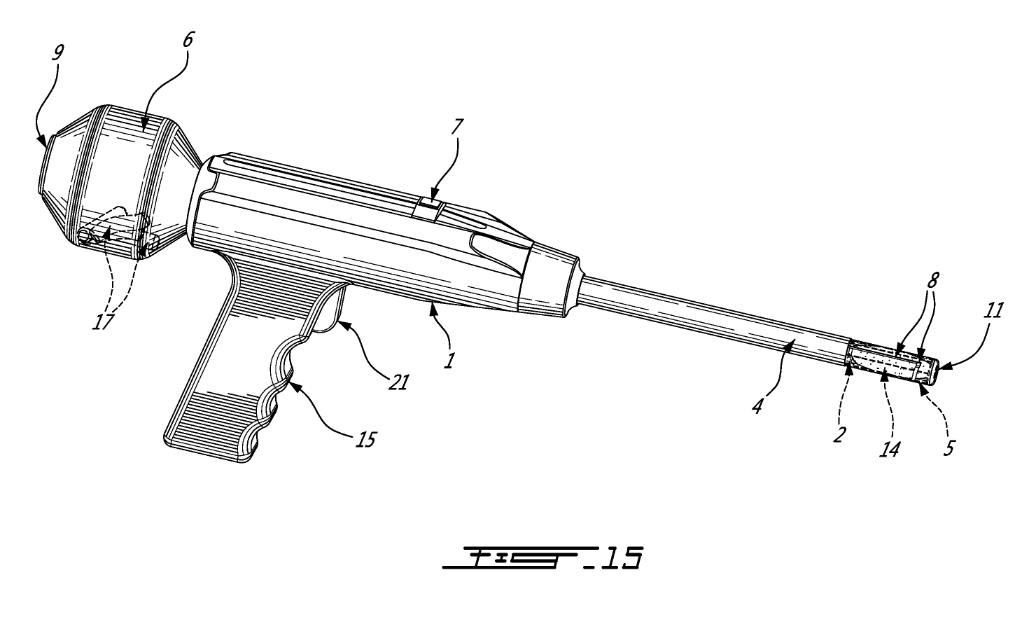

FIG. 15 shows a perspective view of the exemplary morcellator reaching final operation stage and ready to be pulled out of the access port, in accordance with the teachings of the present invention.

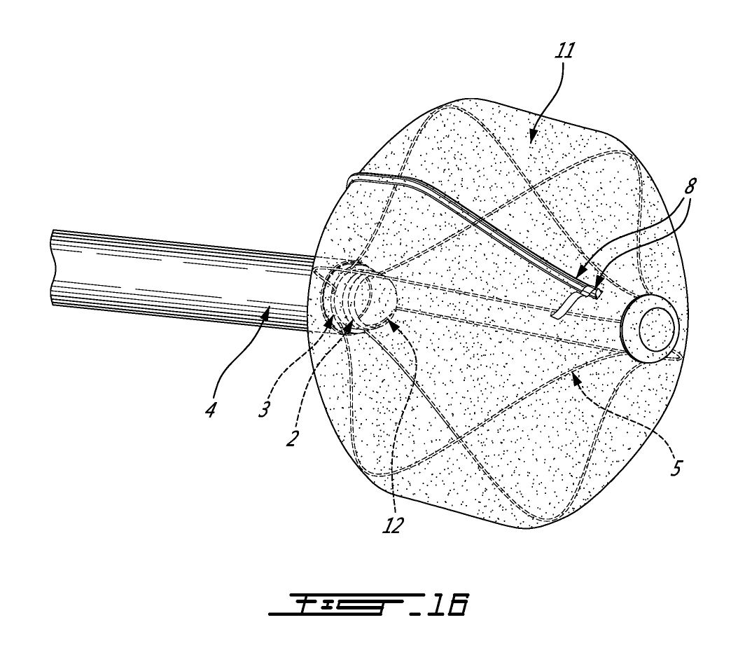

FIG. 16 shows a perspective view of the bag structure showing the inner crest of the cutting tube, in accordance with the teachings of the present invention.

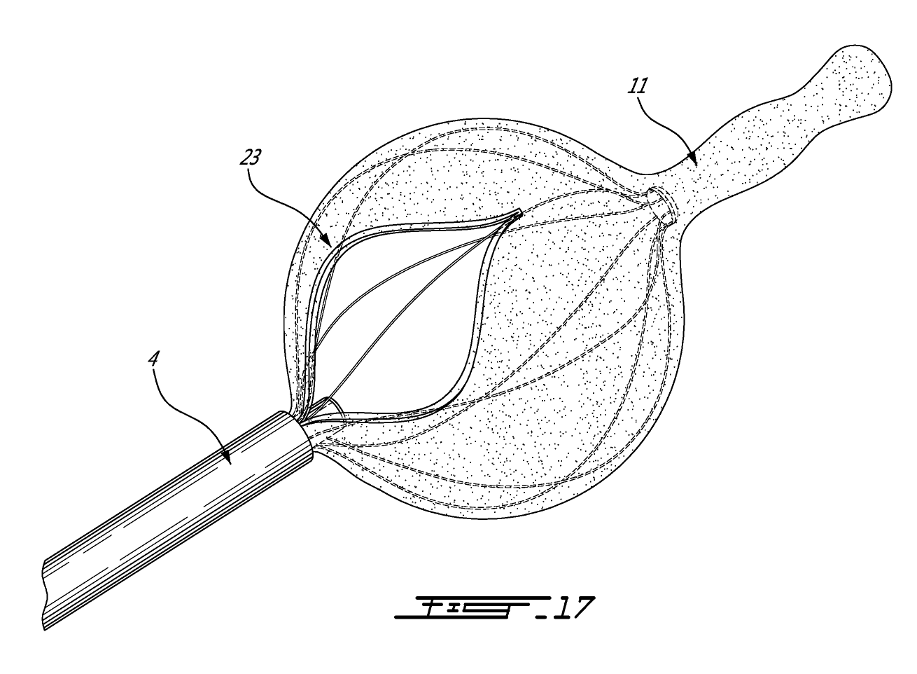

FIG. 17 shows a perspective view of the exemplary morcellator's zipper-like closing mechanism, bag and cage, wherein the bag is significantly longer since this configuration allows the bag to be pulled inside the housing tube to completely seal the bag and proceed to morcellation, in accordance with the teachings of the present invention.

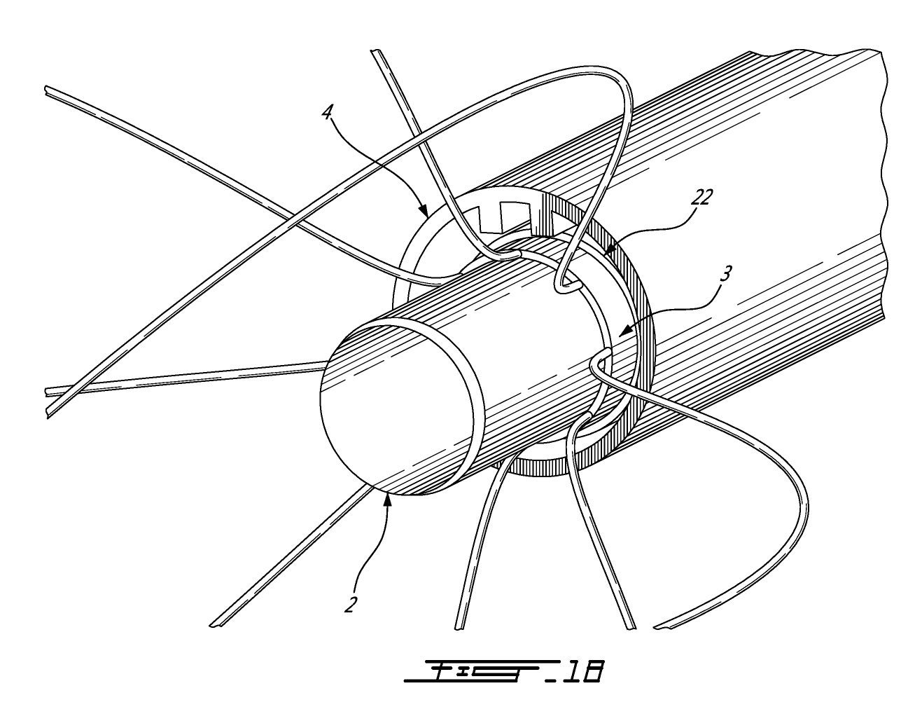

FIG. 18 shows a perspective view of the cutting tube as located inside the cage extension tube in the exemplary morcellator. The housing tube features a groove that is used to close the bag as it is pulled in between the housing tube and cage extension tube. The bag is attached to the bag ring, which is mounted on the cage extension tube and can move freely towards the morcellator's handle, in accordance with the teachings of the present invention.

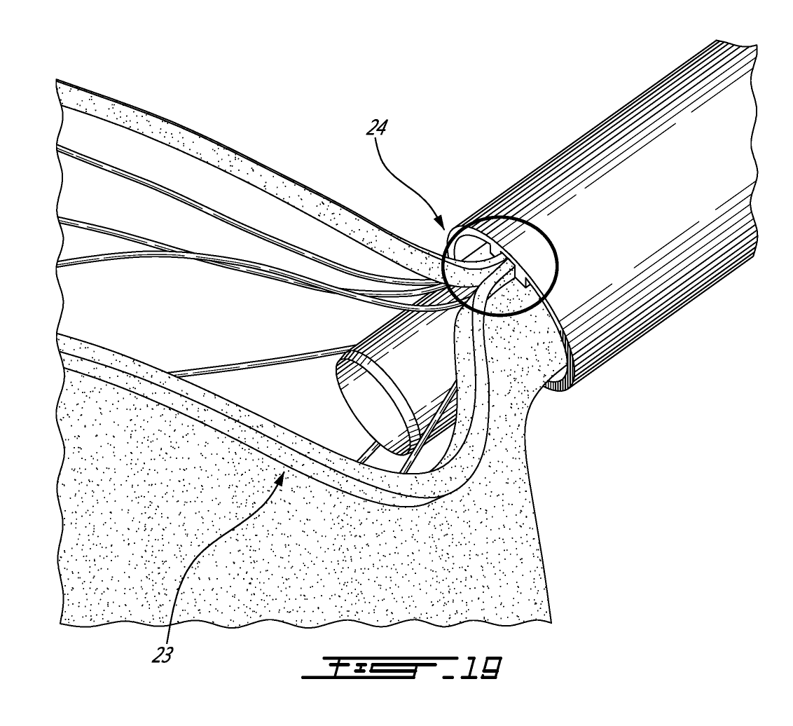

FIG. 19 shows a perspective view of the closing mechanism interacting with the housing tube. With the bag being pulled into the gap between the cage extension tube and the housing tube, the opening is gradually closed until it is completely sealed, in accordance with the teachings of the present invention.

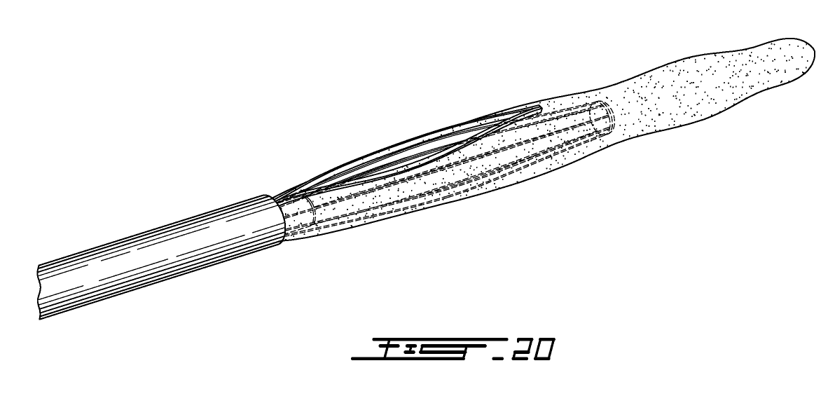

FIG. 20 shows a perspective view of the exemplary morcellator configuration when wrapped into and introducing sleeve. Prior to the introduction of the exemplary morcellator into the body port, the bag and the cage are wrapped into a removable sleeve to keep the cage fully elongated. The sleeve is gradually removed as the elongated cage is introduced in the body, which allows the cage to deploy, in accordance with the teachings of the present invention.

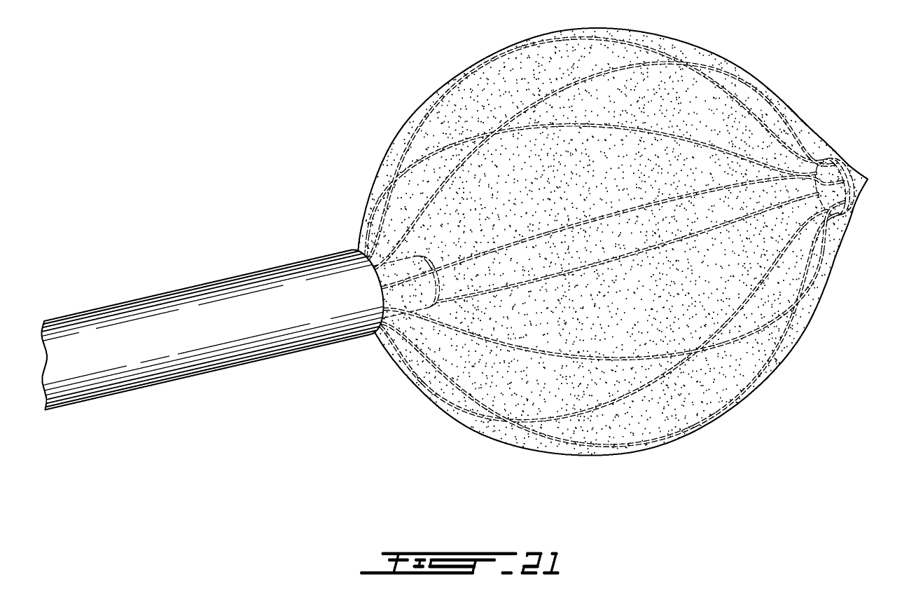

FIG. 21 shows a perspective view of the exemplary morcellator's end when the bag is pulled inside the housing tube with the opening fully closed and sealed. The bag tightly wraps the cage, in accordance with the teachings of the present invention.

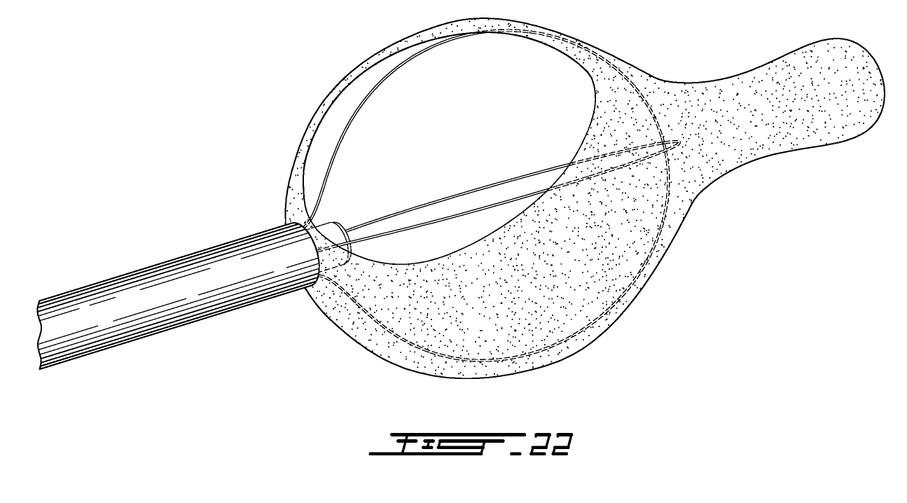

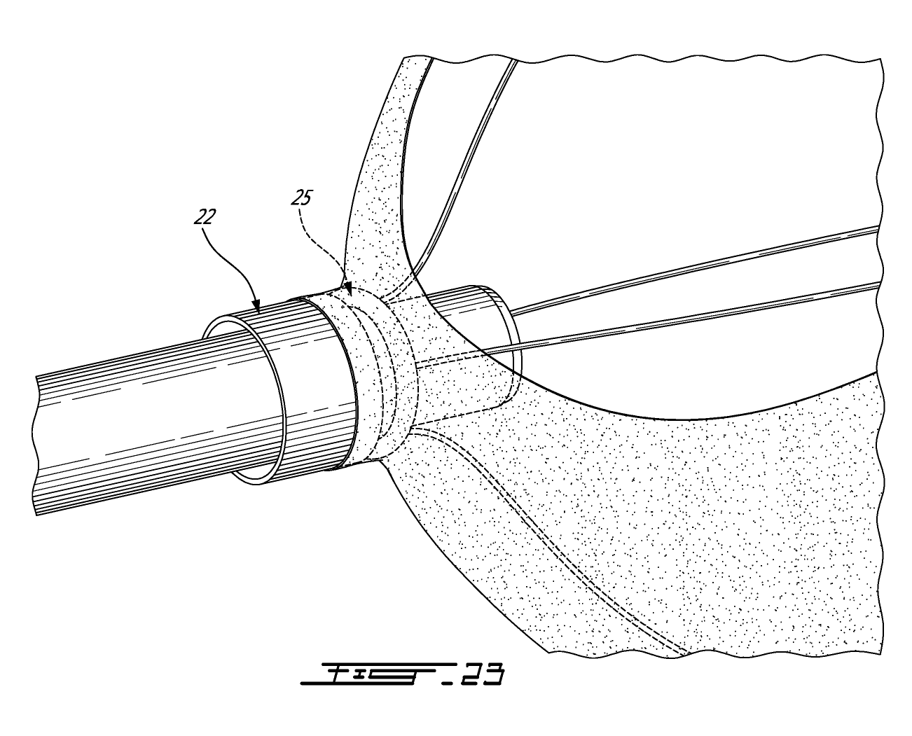

FIG. 22 shows a perspective view of the exemplary morcellator's end showing a simple opening in the bag. The sealing mechanism of the bag is leveraged by pulling the bag between the housing tube and the cage extension tube passed an o-ring located on the cage extension tube, as exemplified on FIG. 23, in accordance with the teachings of the present invention.

FIG. 23 shows a perspective view of the o-ring located on the cage extension tube and in front of the bag ring of the exemplary morcellator. In this manner, when the bag will be pulled inside the housing tube, the bag will be sealed when the opening will have passed the o-ring, in accordance with the teachings of the present invention.

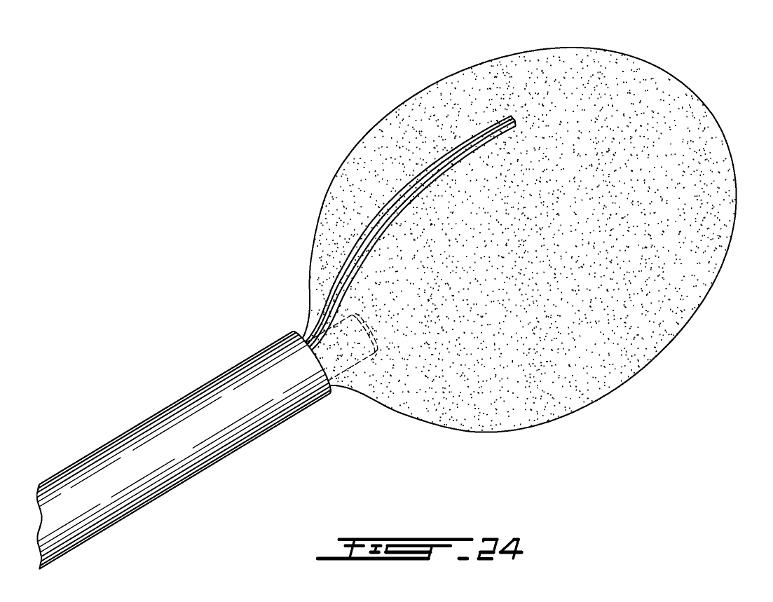

FIG. 24 shows a perspective view of the bag of the exemplary morcellator when inflated, in accordance with the teachings of the present invention.

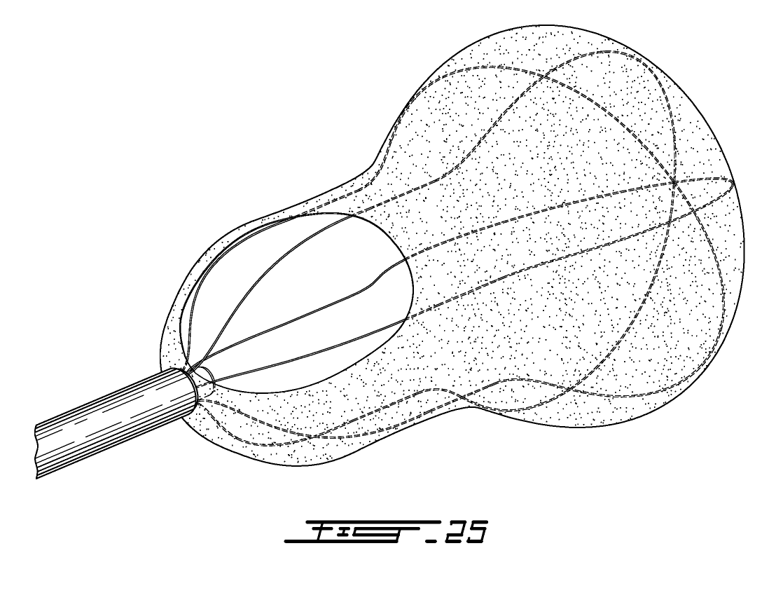

FIG. 25 shows a perspective view of the cage of the exemplary morcellator with two different diameters, with an opening located on the smaller diameter portion of the cage, in accordance with the teachings of the present invention.

FIG. 26 shows a flow chart of an exemplary method 2600 of preventing leakage of severed tissue in a patient's body during morcellation, in accordance with the teachings of the present invention. The method 2600 comprises incising at least one incision in the patient's body 2601, inserting a grasper into the patient's body 2602, grasping a tissue 2603, expanding a sealable enclosure inside the patient's body 2604, receiving the tissue inside the sealable enclosure 2605, sealing the sealable enclosure 2606, engaging a cutter to cut the tissue 2607 and transporting a severed tissue outside the enclosure through a channel toward the outside of the patient's body 2608.

TABLE 1
Exemplary Depicted Mechanism Parts

| Part Number | Part Name |
|---|---|
| (1) | Morcellator |
| (2) | Cutting tube |
| (3) | Cage extension tube |
| (4) | Housing tube |
| (5) | Cage |
| (6) | Collector Reservoir |
| (7) | Manual Cage handle with release |
| (8) | Closure slider mechanism |
| (9) | Lumen |
| (11) | Sheath/Plastic bag |
| (12) | Helical crest |
| (14) | Mass |
| (15) | Handle |
| (17) | Morsel |
| (21) | Trigger |
| (22) | Bag ring |
| (23) | Ziploc closing mechanism |
| (24) | Built-in Ziploc closure |
| (25) | O-ring |

In one embodiment, a morcellator (1) is shown for removing masses as large as 100 cubic centimeters (ml) of tissues during laparoscopic procedures and upgradable for even larger masses. In the depicted embodiment, the morcellator (1) includes a rotating, axially movable cutting member (2) disposed within a rotationally fixed, axially movable outer tube (3). The exemplary outer tube (3) of the morcellator (1) is disposed in the annular space between the cutting member (2) and an outer tube (4) ending with a semi-spherical six-wire control structure (5). In the depicted embodiment, the control structure is encased and sealed by a resealable clear elastic film material (sheath) (11) meant to prevent loss of the severed tissues to the outside, and providing access for the mass to the cavity of the control structure (5) through a leak proof closure mechanism (8). The closure mechanism (8) includes two panel sections on opposite sides along the edges of a 95 mm straight cut on the side of the sheath (11) forming a mouth, it (8) also includes a closure slider mechanism for selectively opening and sealing the mouth using an attached ribbon. The sheath (11) has a round end opening (ring shaped) allowing it to be dressed on the cage structure (5), the round end also has more thickness forming a round lip that slides in a slot on the control structure arm (3) resulting in sealing the sheath. By retracting the inner tube part (3) of the control structure (5) inside the outer tube (4), the confined mass as a result is subjected to a compression force by the control structure (5), locking it in position, preventing the cutting member (2) from twisting the severed tissue, and collapsing the mass to a smaller size after a full cutting stroke; eventually removing the whole mass after several strokes. The morcellator (1) ends with a tissue collector reservoir (6) which ends with a fixed lumen (9) providing access to a laparoscopic grasping instrument. As the cutting member (2) severs tissue, the tissue morsel (17) is drawn proximally into the collector reservoir (6) often by the upward force initiated by the inner helical crest (12) of the rotating cutting tube (2) or by the use of the grasper.

The morcellator (1) is shown to generally include a handle (15), an outer housing tube (4), a control structure (cage) (5), a rotary cutting tube (2), a tissue collector reservoir (6), a resealable sheath (11) and a trigger (21). The handle provides a front aperture (15) through which the outer tube (4), the control structure (cage) (5), the rotary cutting tube (2) extend. At the front aperture (15) the cutting tube is rotatable and slidably received within the tube of the control structure (5). As illustrated the cutting tube (2), cage (5), and housing tube (4) are coaxial and generally tubular in shape.

Preferably, the cutting tube (2) is stainless steel having an outside diameter of about 9.6 mm and a wall thickness of 0.5 mm. The cage (5) is stainless steel with an outside diameter of the tubular part of 11 mm and a wall thickness of 0.7 mm. The confinement part of the cage (5) is made of semi-spherical six-wire structure of stainless steel and 0.4 mm in diameter each. The housing tube (4) is preferably plastic with an outer diameter of 12 mm and a wall thickness of 0.5 mm. The collector reservoir is plastic with a max diameter of 60 mm and a min diameter (inlet) of 13 mm and ending with a fixed lumen with a 13 mm outer diameter.

The cutting tube (2) has a traveling stroke of 130 mm. At the maximum stroke (e.g., 130 mm) the cutter stretches the cage structure (5) to its maximum length causing the wired spring to stretch flat minimizing the diameter of the cage (5) to its minimum diameter possible enabling it to be inserted into the patient through a laparoscopic port site (e.g., a 12 mm size port), or directly through an incision. The morcellator (1) measures around 465 mm wide while fully stretched measuring from the end of the reservoir (6) to the end the cage structure (5).

It is important to note that all measurement and materials mentioned above are subject to variations depending on the size of the mass and manufacturing recommendations (e.g., instead of a cutter (2) fully made out of stainless steel, a plastic tube with a metal cutting edge could be used). For mass sizes above 100 cc, for example, all measurement has to increase accordingly. Moreover, the morcellator can be manufactured for a single use or multiple uses, depending on marketing and manufacturing recommendations, which also can result in changes to measurements and materials used.

The following steps provide an exemplary procedure supported by the present invention:

I. The cutting tube (2) travels to its maximum stroke (e.g., 130 mm) stretching the cage flat while the cage is at its maximum position (e.g., as depicted in the example of FIGS. 3 and 4).

II. The sheath's (11) closure slider is opened to its maximum (inner end) (e.g., as depicted in the example of FIG. 4).

III. The morcellator (1) is then inserted into the patient through a laparoscopic port site.

IV. The cutting tube (2) is then retracted to its minimum position allowing the cage (5) to expand to its semi-spherical shape (e.g., as depicted in the example of FIG. 5).

V. The surgeon locates the mass inside the opened bag structure (5) using his laparoscopic grasper (10) (e.g., as depicted in the example of FIG. 6).

VI. The surgeon closes the sheath's (11) closure slider (travelling to outer end) by using the grasper to pull on the yellow colored ribbon (e.g., as depicted in the example of FIG. 7).

VII. The surgeon presses on the pressure sensitive trigger (21); max press is max Rotation per Minute (RPM) of cutter.

VIII. The cage (5) structure starts retracting inside the outer housing (4) either automatically or manually using the manual cage handle (7), resulting in a compressing force on the mass which locks and traps the mass at the outer end of the cage (e.g., as depicted in the example of FIGS. 9 and 10).

IX. Once the cage (5) retraction stops, the circular cutter starts its first full cutting stroke making a tubular cut through the center of the confined mass (e.g., as depicted in the example of FIG. 11).

X. The morsel is then drawn to the collector reservoir (6). The tissue is drawn proximally into the collector reservoir often by the upward force initiated by the inner helical crest (12) of the rotating cutting tube (2) or by the use of the grasper (e.g., as depicted in the example of FIG. 16).

XI. After the cutter (2) retracts to its minimum position, the continuous pulling force on the cage's extension (3) tube causing the compression force on the mass will result in the collapse of the mass (14) to a smaller size and diameter (e.g., as depicted in the example of FIGS. 12 and 13).

XII. As long as the trigger (21) is pressed, the cutter will keep oscillating back and forth while rotating at desired RPM speed (controlled by the pressure on the trigger).

XIII Step IX is repeated until the mass is morcellated to a minimum size (e.g., as depicted in the example of FIGS. 14 and 15).

XIV. Once the mass reaches minimum size, the cage structure (5) will have the required clearance to fully retract inside the outer housing tube (4) or to be safely pulled out of the access port.

XV. The morcellator (1) is withdrawn out of the laparoscopic port ending the operation.

XVI. During the operation of the morcellator (1), the cage (5) and its extension tube (3) can be released from the body of the morcellator (1) by pressing on the release button and turning the handle (7) at a 90 degree angle. This is helpful if the surgeon requires clearing the space while the bag is expanded with the mass (14) inside. It also helps to rotate the access part of the bag (8) at any angle required.

XVII. At any point, the collector reservoir (6) can be exchanged with a new one during the operation of the morcellator (1).

In some embodiments, the device provides the possibility to seal the bag and/or enclosure means after tissues are introduced in it. In one embodiment, the bag may be pulled inside the shaft in order to shut the opening and, therefore, prevent tissues from being ejected out of the bag. The concept presented here may rely on a bag featuring a zipper-like type closing system proximal to the tip of the morcellator, see FIG. 17. By pulling the bag inside the morcellator's shaft, the opening may close and seal with no possibility to re-open the bag. The bag length exceeds the cage length, but it will perfectly fit on the cage when the open portion of the bag will be inserted into the morcellator's shaft.

In one embodiment, as shown in FIG. 18, the rotating blade is located inside a fixed cylindrical part (stator) to which the cage is mounted. In this embodiment, the cage is not moving; only the bag can slide inside the morcellator's shaft. Also, the blade is not advancing but graspers are used to bring tissues at the rotating blade and take them out of the morcellator's shaft. The blade protrudes from the tip of the outer shell to help with cutting the tissues. The outer shell features a groove that is used to close the bag as it is introduced into the shaft between the outer shell and the stator. The bag may be attached to a ring (bag ring) mounted on the stator and it can move freely towards the morcellator's handle.

Optionally, the blade may also advance in this embodiment. In this case, the cage is moving, and once the bag slides inside the outer tube sealing it, the cage collapses on the mass by moving into the outer tube and the morcellation begins.

FIG. 19 shows the details of the closing system interacting with the outer shell of the morcellator's shaft. Optionally, as the bag is pulled into the gap between the cage extension tube/stator and the housing tube/outer shell, the opening is gradually closed until it is completely sealed.

In this embodiment, prior to the introduction of the morcellator into the body port, the bag and the cage are wrapped using a sleeve to keep the cage fully elongated, see FIG. 20. Once the tip of the morcellator is introduced in the abdomen, the sleeve is removed and the cage deploys with the bag open as shown in FIG. 17. Tissues are then put in the bag using the graspers. When full, the bag is closed by pulling on the bag ring, which also tightly wraps the cage with the bag, see FIG. 21. An advantage of this embodiment would be the ability to close the bag using a sliding handle on the body of the device instead of using the grasper internally to close the slider mechanism.

FIG. 24 shows an embodiment where instead of using a cage to support the bag/sheath, a positive pressure is applied to maintain the bag inflated during morcellation. For example, pressurized CO₂ may be provided through the housing tube. When the bag is introduced in the body, it may be deflated with an opening, and may feature a zipper-like mechanism in an open position. Tissues are then put in the bag and closed. The bag could then be inflated and morcellation could begin. A potential advantage of this embodiment is that the device may have the smallest outer housing diameter possible, maximizing the size of the morcels. The foregoing may result in a reduced operating duration. Furthermore, this embodiment may use the least amount of parts possible. This embodiment may also comprise a disposable device, making the device cost effective since it is intended to be a one-time use device. The minimal body size/length may result in a less bulky, light weight device with simpler mechanics. This may result in a lower mass production cost/unit.

Further potential advantages to the embodiments depicted in FIGS. 1 through 16 include ease of use by the surgeon and the elimination of errors. The bag can be easily reopened to add more tissues if required by the operator and closed. Operating the device may be very simple, and require minimal training, if any. The design makes it difficult to cut through the bag, minimizing human error, and making it difficult to leave any tissue traces behind. These embodiments may enable a clear visual field during the procedure. These embodiments may also include a rotatable bag system.

In another embodiment, there may be an opening in the bag that could be sealed using an o-ring located inside the shaft, see FIG. 22. The cage is also simplified in this embodiment.

The location of the o-ring is illustrated in FIG. 23. The o-ring may be located around the stator and in front of the bag ring. In this manner, when the bag will be pulled inside the shaft/housing tube, the bag will be sealed when the opening will have passed the o-ring.

Optionally, the cutter may not be advancing in the exemplary morcellator in the previous embodiment. In this case, the number of struts may be reduced to a minimum since the blade is not advancing and graspers are used to introduce tissues into the rotating blade. A sleeve, optionally made of plastic, may be used to reduce the profile of the cage prior to introducing the morcellator into the patient, see FIG. 20. Once the tip of the morcellator is introduced in the abdomen, the sleeve is removed and the cage deploys with the bag fully open as shown in FIG. 22. Tissues are then put in the bag using the graspers. When full, the bag is closed by pulling on the bag ring, which also tightly wrap the cage with the bag, see FIG. 21.

The advantages of the foregoing embodiment may include the elimination of the manual closing of the slider, which accordingly may speed the process and the ease of operating. The embodiment will also facilitate an ease of manufacturing and a further elimination of errors.

Optionally, the cage mounted on the tip of the morcellator's shaft may be removed and simply a positive pressure is applied inside the bag. An inflated bag would keep the bag membrane away from the rotating blade and would offer very good visibility to the surgeon. Pressurized CO₂ could be provided through the outer shell of the shaft. This embodiment may be simple to use and manufacture, and may have few mechanical system requirements. This embodiment may be best for soft tissue tumors.

Optionally, the exemplary morcellator could use a very stretchable bag that would be sitting at the tip of the cage. After tissues are put inside the cage, the bag would be rolled around the cage all the way up to the shaft to make a tight seal. Optionally, this bag could be condom-shaped. Optionally, the bag may roll in an outward direction from the tube, and may be sealed with a clip or a clip-like mechanism.

Optionally, a balloon may be used to seal the opening of the bag after the bag is filled with tissues. A catheter balloon could be introduced in the patient after the bag is filled, and precisely put at the bag opening, and inflated to make a tight seal.

In the course of the above described embodiments, a number of alternatives have been identified and others may well occur to those skilled in the art without departing from the field of the invention. Thus, various combinations, sub-combinations, and sundry adaptations are maintained under the principles of the provided invention.

A method is generally conceived to be a self-consistent sequence of steps leading to a desired result. Skilled persons will readily understand how the steps of the exemplary methods described herein could be rearranged without affecting the desired result. Exemplary embodiments have been described to demonstrate the use, principles, and function of the invention disclosed herein.

The description of the present invention has been presented for purposes of illustration but is not intended to be exhaustive or limited to the disclosed embodiments. Many modifications and variations will be apparent to those of ordinary skill in the art. The embodiments were chosen to explain the principles of the invention and its practical applications and to enable others of ordinary skill in the art to understand the invention in order to implement various embodiments with various modifications as might be suited to other contemplated uses.

What is claimed is:

1. A device for secured retrieval of tissue from inside a patient body, the device comprising:
a handle for controlling the device, the handle being configured for manipulation of the device from outside of the patient body and comprising a specimen discharging aperture towards the outside of the patient body;
a tube defining a fluid communication channel between an inward end and an outward end extending towards the specimen discharging aperture of the handle, the tube comprising an external surface configured on at least a portion thereof to interface between the inside and the outside of the patient body therealong;
a movable cutter, housed within the tube, controllably extendable from the inward end of the tube while allowing continued fluid communication in the fluid communication channel;
a retractable enclosure at least partly housed within the tube comprising:
a supporting frame that defines a first retracted configuration and a second expanded configurations, wherein the first retracted configuration defines a first enclosure volume and hinders the fluid communication in the fluid communication channel;
an impermeable membrane, linked to the supporting frame, comprising an opening;
a frame actuator mechanism that controls amplitude of the supporting frame between the first retracted configuration and the second expanded configuration and vice-versa;
wherein the retractable enclosure, in the second expanded configuration, extends from the inward end of the tube and has a variable second enclosure volume, greater than or equal to the first enclosure volume, defined by the frame actuator mechanism upon controlling the amplitude of the supporting frame; and
a sealing mechanism, for controllably sealing the opening in the impermeable membrane, configured to:
unseal the opening when the second enclosure volume is greater than the first enclosure volume; and
when the tissue is positioned within the enclosure, sealably close the opening in the impermeable membrane, thereby sealing the retractable enclosure;
wherein the retractable enclosure receives the moveable cutter for morcellating the tissue into tissue specimens when the opening in the impermeable membrane is sealably closed, whereby the tissue specimens are able to transit in the fluid communication channel towards the discharging aperture after morcellation; and
wherein the frame actuator mechanism reduces the amplitude of the supporting frame causing the tissue specimens to be compressed by the supporting frame during transition from the second expanded configuration to the first retracted configuration, the tissue specimens being discharged through the fluid communication channel towards the discharging aperture.

2. The device of claim 1, wherein the sealing mechanism comprises a zipper-like mechanism.

3. The device of claim 1, further comprising a tissue collection reservoir for receiving the severed tissue through the specimen discharging aperture on the outside of the patient's body.

4. The device of claim 1, wherein the tissue specimens are compressed by the supporting frame and the impermeable membrane.

5. The device of claim 1, wherein the tissue specimens are compressed by the supporting frame while the impermeable membrane defines a membrane volume greater than the second volume.

6. The device of claim 1, wherein the sealing mechanism unseals the opening only when the second enclosure volume is greater than the first enclosure volume.

7. The device of claim 1, wherein the sealing mechanism, when the second enclosure volume is equal to the first enclosure volume, maintains closed the opening in the impermeable membrane and prevents unsealing of the opening in the impermeable membrane.

8. A method for secured retrieval of tissue from inside a patient body, the method comprising:
inserting a device from outside of the patient body, the device comprising a specimen discharging aperture towards the outside of the patient body and a tube defining a fluid communication channel between an inward end and an outward end extending towards the specimen discharging aperture, the tube comprising an external surface configured on at least a portion thereof to interface between the inside and the outside of the patient body therealong;
from the device, expanding a retractable enclosure inside the patient's body, the retractable enclosure being at least partly housed within the tube and comprising an impermeable membrane comprising an opening, the expanding of the retractable enclosure comprising:
extending a supporting frame from the inward end of the tube into an expanded configuration from a retracted configuration, wherein the retracted configuration defines a first enclosure volume and hinders the fluid communication in the fluid communication channel and wherein the expanded configuration has a variable second enclosure volume, greater than or equal to the first enclosure volume, defined by the frame actuator mechanism upon controlling the amplitude of the supporting frame;
unsealing the opening when the second enclosure volume is greater than the first enclosure volume;
when the tissue is positioned within the enclosure, sealably closing the opening in the impermeable membrane, thereby sealing the retractable enclosure;
controllably extending a movable cutter, housed within the tube, from the inward end of the tube while allowing continued fluid communication in the fluid communication channel, wherein the moveable cutter morcellates the tissue into tissue specimens when the opening in the impermeable membrane is sealably closed, whereby the tissue specimens are able to transit in the fluid communication channel towards the discharging aperture after morcellation; and
reducing the amplitude of the supporting frame causing the tissue specimens to be compressed by the supporting frame during transition from the second expanded configuration to the first retracted configuration, the tissue specimens being discharged through the fluid communication channel towards the discharging aperture.

9. The method of claim 8, wherein sealing and unsealing are performed using a zipper-like sealing mechanism.

10. The method of claim 8, further comprising discharging the severed tissue into a tissue collection reservoir through the specimen discharging aperture on the outside of the patient's body.

11. The method of claim 8, wherein the tissue specimens are compressed by the supporting frame and the impermeable membrane.

12. The method of claim 8, wherein the tissue specimens are compressed by the supporting frame while the impermeable membrane defines a membrane volume greater than the second volume.

13. The method of claim 8, wherein unsealing the opening is performed only when the second enclosure volume is greater than the first enclosure volume.

14. The method of claim 8, wherein, when the second enclosure volume is equal to the first enclosure volume, the opening in the impermeable membrane is maintained closed and prevented from unsealing.

15. A kit for secured retrieval of tissue from inside a patient body comprising:
a device comprising:
a handle for controlling the device, the handle being configured for manipulation of the device from outside of the patient body and comprising a specimen discharging aperture towards the outside of the patient body;
a tube defining a fluid communication channel between an inward end and an outward end extending towards the specimen discharging aperture of the handle, the tube comprising an external surface configured on at least a portion thereof to interface between the inside and the outside of the patient body therealong;
a movable cutter, housed within the tube, controllably extendable from the inward end of the tube while allowing continued fluid communication in the fluid communication channel;
a retractable enclosure at least partly housed within the tube comprising:
a supporting frame that defines a first retracted configuration and a second expanded configurations, wherein the first retracted configuration defines a first enclosure volume and hinders the fluid communication in the fluid communication channel;
an impermeable membrane, linked to the supporting frame, comprising an opening;
a frame actuator mechanism that controls amplitude of the supporting frame between the first retracted configuration and the second expanded configuration and vice-versa;
wherein the retractable enclosure, in the second expanded configuration, extends from the inward end of the tube and has a variable second enclosure volume, greater than or equal to the first enclosure volume, defined by the frame actuator mechanism upon controlling the amplitude of the supporting frame; and
a sealing mechanism, for controllably sealing the opening in the impermeable membrane, configured to:
unseal the opening when the second enclosure volume is greater than the first enclosure volume; and
when the tissue is positioned within the enclosure, sealably close the opening in the impermeable membrane, thereby sealing the retractable enclosure;
wherein the retractable enclosure receives the moveable cutter for morcellating the tissue into tissue specimens when the opening in the impermeable membrane is sealably closed, whereby the tissue specimens are able to transit in the fluid communication channel towards the discharging aperture after morcellation; and
wherein the frame actuator mechanism reduces the amplitude of the supporting frame causing the tissue specimens to be compressed by the supporting frame during transition from the second expanded configuration to the first retracted configuration, the tissue specimens being discharged through the fluid communication channel towards the discharging aperture; and
a grasper.

16. The kit of claim 16, wherein the device and the grasper are disposable.

17. The kit of claim 16, wherein only the grasper is disposable and the device is reusable.
